(19) United States
(12) Patent Application Publication
(10) Pub. No.: US 2017/0065835 A1
(43) Pub. Date: Mar. 9, 2017

(54) HELMET-TYPE LOW-INTENSITY FOCUSED ULTRASOUND STIMULATION DEVICE AND SYSTEM

(71) Applicant: MSP CO., LTD, Incheon (KR)

(72) Inventor: Moon Seo Park, Incheon (KR)

(73) Assignee: MSP CO., LTD, Incheon (KR)

(21) Appl. No.: 15/121,841

(22) PCT Filed: Feb. 27, 2015
(86) PCT No.: PCT/KR2015/001918

§ 371 Date: Aug. 26, 2016

(30) Foreign Application Priority Data
Feb. 28, 2014 (KR) 10-2014-0024368
Feb. 28, 2014 (KR) 10-2014-0024369
Feb. 28, 2014 (KR) 10-2014-0024370

(51) Int. Cl. A61N 7/00; A61B 90/00; A61B 5/00; A61B 6/00; A61B 5/055; A61B 6/03

(52) U.S. Cl. CPC A61N 7/00; A61B 5/055; A61B 6/032; A61B 5/0042; A61B 6/501; A61B 90/39; A61B 5/0077; A61N 2007/0026; A61B 2090/3937

(57) ABSTRACT

A helmet-type low-intensity focused ultrasound stimulation device includes: a first guide part formed in an arc shape and provided in a longitudinal direction; a second guide part which is formed in an arc shape and is connected in a transverse direction so as to be orthogonal to the first guide part; an ultrasound module which is connected to the second guide part and includes a transducer for generating ultrasound waves moving towards an inner direction; and a support part which is worn on a head of a user, and to which the first guide part is fixed and both end portions of the second guide part are rotationally fixed, in which the second guide part rotates in the longitudinal direction by allowing one point thereof to be guided along the first guide part, and the transducer is movable along the second guide part in a transverse direction.

### TECHNICAL FIELD

The present invention relates to a helmet-type low-intensity ultrasound stimulation device, and more particularly, to a device for stimulating a head of a user by using focused ultrasonic waves.

### BACKGROUND ART

A method for controlling a brain function includes a brain cell activating method through medication, an electric stimulation method using a probe, and a transcranial magnetic stimulation method.

However, the brain cell activating method through medication has a problem in that it is impossible to control only a desired specific portion through medication, and the electric stimulation method using a probe may temporarily or permanently changing a function of the brain by applying an electric stimuli by implanting or inserting a probe into a specific position of the brain by electrocorticogram or Deep Brain Stimulation (DBS) used in a brain surgery, but has a risk according to an invasive method. Further, the transcranial magnetic stimulation method is a non-invasive method controlling a brain function, and is a method of adjusting a brain function by generating a strong magnetic field at an outside of a skull and allowing an inductive current to flow in a cerebrocortex, but in a case of a transcranial magnetic stimulation device, an area of the non-invasively stimulated cerebrocortex is wide (2 to 3 cm or more), and only a skull and a surface of the cerebrocortex in a depth of 1 to 2 cm can be stimulated and an intensity of the magnetic field is sharply decreased in an area deeply located inside the brain, so that it is difficult to precisely control the brain function.

### DISCLOSURE

### Technical Problem

The present invention provides a structure, which is capable of precisely controlling a position of a transducer in a focused ultrasound stimulation device using ultrasound waves that is a non-invasive method.

Further, the present invention provides a helmet-type low-intensity focused ultrasound stimulation device having a structure enabling ultrasound waves to be easily transmitted.

Further, the present invention provides a helmet-type low-intensity focused ultrasound stimulation system, which is capable of easily performing a focused ultrasound stimulation by managing a stimulation position of ultrasound waves including a depth and an ultrasound stimulation method as one sequence.

Further, the present invention provides a helmet-type low-intensity focused ultrasound stimulation system, in which an ultrasound stimulation sequence may be automatically performed on a patient according to a lesion.

Further, the present invention provides a helmet-type low-intensity focused ultrasound stimulation system, which is capable of precisely radiating ultrasound waves by tracking a change of a wearing state of a patient even when the wearing state of the patient is slightly changed, and reflecting the tracked change to a position control of a transducer.

Further, the present invention provides a helmet-type low-intensity focused ultrasound stimulation system, which is capable of precisely radiating ultrasound waves by using an MRI image and the like provided from an external device.

### Technical Solution

A helmet-type low-intensity focused ultrasound stimulation device according to the present invention includes: a first guide part which is formed in an arc shape and is provided in a longitudinal direction; a second guide part which is formed in an arc shape and is connected in a transverse direction so as to be orthogonal to the first guide part; an ultrasound module which is connected to the second guide part and includes a transducer for generating ultrasound waves moving towards an inner direction; and a support part which is worn on a head of a user, and to which the first guide part is fixed and both end portions of the second guide part are rotationally fixed, in which the second guide part rotates in the longitudinal direction by allowing one point thereof to be guided along the first guide part, and the transducer is movable along the second guide part in a transverse direction.

The ultrasound module may include a distance adjusting unit which moves the transducer in the moving direction of the ultrasound waves or in a reverse direction.

The first guide part may be formed with a first rack gear in the longitudinal direction, and the second guide part may be provided with a first pinion corresponding to the first rack gear.

The second guide part may include an extended part which protrudes in an upper direction and include a first motor driving the first pinion at an inner side thereof

The second guide part may be formed with a second rack gear in the longitudinal direction, and the ultrasound module may include a second pinion corresponding to the second rack gear.

The ultrasound module may include a fixing part which includes a second motor driving the second pinion at an inner side thereof.

The distance adjusting unit may include: a third rack gear formed from the fixing part in an inner direction; a third pinion which moves in the inner direction or an outer direction in response to the third rack gear and adjusts a radial distance of the transducer; and a third motor which drives the third pinion.

The helmet-type low-intensity focused ultrasound stimulation device may further include a transmitting unit which stores an ultrasound medium material at an inner side thereof, and is interposed between the head of the user and the transducer to mediate ultrasound transmission.

The transmitting unit may be fixed to a lower end of the transducer.

The transmitting unit may be formed of a synthetic resin material.

The medium material may be de-gas water.

In the meantime, a helmet-type low-intensity focused ultrasound system according to the present invention includes: a brain map database which stores a 3D relative coordinate value for each part of a head of a standard human including a brain; a sequence database which stores an ultrasound stimulation method including at least one of an intensity of ultrasound stimulation, an ultrasound stimulation time, the number of times of the ultrasound stimulation, a period of the ultrasound stimulation, and sequence data related to a stimulation control including a relative coordinate value of a specific part of a brain to which the ultrasound stimulation method is to be applied; a helmet-type low-intensity focused ultrasound device which includes a transducer for generating ultrasound waves, and a support part mounted on a head of a patient and supporting the transducer to have a position movement on the head of the patient; and a sequence control means which controls a position of the transducer and controlling an operation of the transducer at the corresponding position according to relative coordinates and a stimulation method corresponding to corresponding sequence data stored in the sequence database by selecting any one of the sequence data.

The sequence database may further include sequence data formed of a combination of sets including the ultrasound stimulation method and the relative coordinate value.

The sequence data may be stored in the sequence database in correspondence with a specific treatment including any one of relief and a treatment of a specific disease and relief and a treatment of a specific pain.

The sequence control means may receive data for a specific treatment from the outside, inquires sequence data corresponding to the specific treatment in the sequence database, and control a position and an operation of the transducer according to the inquired sequence data.

The helmet-type low-intensity focused ultrasound stimulation system may further include a matching means which receives image data of the head of the patient from an external device performing any one photographing method selected from Computerized Tomography (CT), Magnetic Resonance Imaging (MRI), and Functional Magnetic Resonance Imaging (fMRI), and matches the received image data with the 3D relative coordinate value of the head of the standard human body stored in the brain map database.

The sequence control means may control a position of the transducer by using a relative coordinate value, which is converted so as to correspond to the head of the patient by the matching means.

In the meantime, a helmet-type low-intensity focused ultrasound stimulation system according to the present invention includes: a helmet-type low-intensity focused ultrasound stimulation device which includes a transducer for generating ultrasound waves, and a support part mounted on a head of a patient and supporting the transducer to have a position movement on the head of the patient; and a position setting means which sets initial coordinates of the transducer; a sequence control means which performs a position control based on the initial coordinates of the transducer, and performs a control according to an ultrasound stimulation method including at least one of an intensity of a stimulation, a stimulation time, the number of times of the stimulation, a period of the stimulation of ultrasound waves generated by the transducer; one or more optical cameras which is fixed to the helmet-type low-intensity focused ultrasound stimulation device, and photographs third markers attached onto the head of the patient; and a position correcting means which obtains position change information of the helmet-type low-intensity focused ultrasound stimulation device for the head of the patient based on a position change of the third marker on an image photographed by the optical camera.

The helmet-type low-intensity focused ultrasound stimulation system may further include: a brain map database which stores a 3D relative coordinate data for each part of a head of a standard human body including a brain; and a matching means which receives image data of the head of the patient from an external device performing any one photographing method selected from Computerized Tomography (CT), Magnetic Resonance Imaging (MRI), and Functional Magnetic Resonance Imaging (fMRI), and matches the received image data with the 3D relative coordinate value of the head of the standard human body stored in the brain map database, in which the position setting means sets initial coordinates of the transducer by linking the matched coordinate system of the image data of the head of the patient with a coordinate system for a control area of the transducer.

The matching means may receive the image data of the head of the patient, to which a plurality of first markers detectible by the selected photographing method is attached, from the external device, the optical camera may photograph a second marker provided at a position of the first marker, and the position setting means may link the matched coordinate system of the image data of the head of the patient and the coordinate system for the control area of the transducer based on the positions of the first marker and the second marker.

The position setting means may calculate a movement distance of any one marker, which is selected as a reference marker among the third markers, according to a position change between specific frames of an image photographed by the optical camera, and calculate a rotation angle of one or more markers, which are not selected as the reference marker among the third markers, based on the reference marker.

The position correcting means may transmit the calculated movement distance of the reference marker and the calculated rotation angle based on the reference marker to the position setting means, and the position setting means may reset the initial coordinates of the transducer by reflecting the received movement distance of the reference marker and the received rotation angle based on the reference marker.

The position correcting means may transmit the calculated movement distance of the reference marker and the calculated rotation angle based on the reference marker to the sequence control means, and the sequence control means may reflect the received movement distance of the reference marker and the received rotation angle based on the reference marker during a position control of the transducer.

The second marker and the third marker may be photoreflective.

The first marker may be attached to each of the top of the head, a forehead, an occipital area adjacent to an ear of the patient.

The second marker may be attached to each of the forehead and the occipital area adjacent to the ear at least.

The second marker may be attached onto the first marker after an image is photographed by the external device.

The third markers may include two or more markers selected from the second marker.

### Advantageous Effects

According to the present invention, the focused stimulation apparatus using ultrasound waves that is a non-invasive method adopts a structure, in which a position of a transducer uses a polar coordinate system or a spherical coordinate system similar to the polar coordinate system, so that it is possible to precisely control a position of the transducer.

Further, according to the present invention, there are provided the compact transmission unit and the like, which may be directly worn on a head of a patient or may be attached to a transducer and move together with the transducer, so that it is possible to precisely transmit ultrasound waves to a target part without an interposition of air during an radiation of ultrasound waves.

Further, according to the present invention, a stimulation position of ultrasound waves including a depth and an ultrasound stimulation method may be managed as one sequence, and further, a plurality of sequences may be combined and automatically applied to a patient, so that it is possible to easily perform a focused ultrasound stimulation.

Further, according to the present invention, an ultrasound stimulation sequence may be automatically performed on a patient according to a lesion, so that it is possible to easily perform an ultrasound stimulation on the patient.

Further, according to the present invention, even when a wearing state of the patient is slightly changed because a patient moves in a state where the patient wears the helmet-type low-intensity focused ultrasound stimulation device or external force is applied to the helmet-type low-intensity focused ultrasound stimulation device, it is possible to track the change and reflect the tracked change to a position control of a transducer, thereby precisely radiating ultrasound waves.

Further, according to the present invention, it is possible to reflect an individual characteristic and precisely radiate ultrasound waves by receiving an image of CT, MRI, and fMRI provided from an external device and matching the received image to relative coordinates of a head of a standard human body.

Further, according to the present invention, it is possible to precisely radiate ultrasound waves by linking matched coordinates of a personal brain structure with a spatial coordinate system for controlling a transducer.

### DESCRIPTION OF DRAWINGS

FIG. 1 is a perspective view illustrating a helmet-type low-intensity focused ultrasound stimulation device according to an exemplary embodiment of the present invention.

FIG. 2 is a front view illustrating the helmet-type low-intensity focused ultrasound stimulation device according to the exemplary embodiment of the present invention.

FIG. 3 is a side view illustrating the helmet-type low-intensity focused ultrasound stimulation device according to the exemplary embodiment of the present invention.

FIG. 4 is a partially exploded perspective view illustrating a part of the helmet-type low-intensity focused ultrasound stimulation device according to the exemplary embodiment of the present invention.

FIG. 5 is a longitudinal cross-sectional view taken along line A-A of FIG. 1.

FIG. 6 is a longitudinal cross-sectional view for describing an ultrasound module.

FIG. 7 is a side view illustrating the helmet-type low-intensity focused ultrasound stimulation device of FIG. 6.

FIG. 8 is a perspective view illustrating a longitudinal location control of the helmet-type low-intensity focused ultrasound stimulation device according to the exemplary embodiment of the present invention.

FIG. 9 is a perspective view illustrating a transverse location control of the helmet-type low-intensity focused ultrasound stimulation device according to the exemplary embodiment of the present invention.

FIG. 10 is a diagram schematically illustrating a distance control of an ultrasound module according to the exemplary embodiment of the present invention.

FIG. 11 is a perspective view illustrating a distance control of the ultrasound module according to the exemplary embodiment of the present invention.

FIG. 12 is a perspective view illustrating a helmet-type low-intensity focused ultrasound stimulation device including an ultrasound module according to another exemplary embodiment of the present invention.

FIG. 13 is a cross-sectional view illustrating the ultrasound module according to the exemplary embodiment of FIG. 12.

FIG. 14 is a block diagram illustrating a helmet-type low-intensity focused ultrasound stimulation system according to an exemplary embodiment of the present invention.

FIGS. 15 and 16 are diagrams schematically illustrating an attachment position of a first marker according to the exemplary embodiment.

FIG. 17 is a diagram schematically illustrating goggles, to which a third marker is attached.

FIG. 18 is a diagram schematically illustrating a state where a patient wears goggles.

FIGS. 19 and 20 are diagrams schematically illustrating the states, in which third markers in a front surface and a side surface are photographed.

FIGS. 21 and 22 are diagrams schematically illustrating an example of continuous frames of the photographed third marker.

### BEST MODE

A helmet-type low-intensity focused ultrasound stimulation device according to the present invention includes: a first guide part which is formed in an arc shape and is provided in a longitudinal direction; a second guide part which is formed in an arc shape and is connected in a transverse direction so as to be orthogonal to the first guide part; an ultrasound module which is connected to the second guide part and includes a transducer for generating ultrasound waves moving towards an inner direction; and a support part which is worn on a head of a user, and to which the first guide part is fixed and both end portions of the second guide part are rotationally fixed, wherein the second guide part rotates in the longitudinal direction by allowing one point thereof to be guided along the first guide part, and the transducer is movable along the second guide part in a transverse direction.

### [Mode for Carrying Out the Invention]

Hereinafter, exemplary embodiments of the present invention will be described with reference to the accompanying drawings. Unless there is a special definition or mention, terms indicating a direction used in the present description are based on a state illustrated in the drawing. Further, the same reference numeral designates the same member throughout each exemplary embodiment. In the meantime, for convenience of the description, a thickness or a size of each constituent element illustrated in the drawings may be exaggerated, and it does not mean that the constituent element needs to be actually configured with a corresponding size or a ratio between the elements.

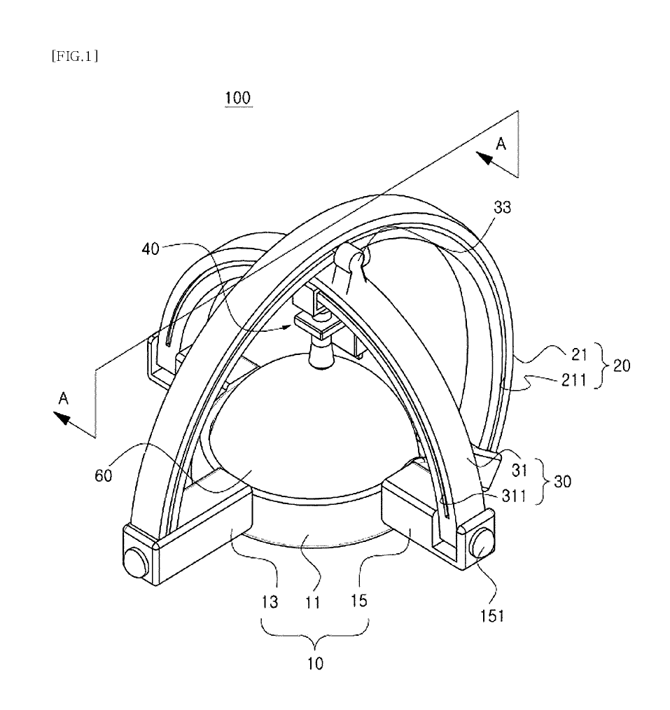

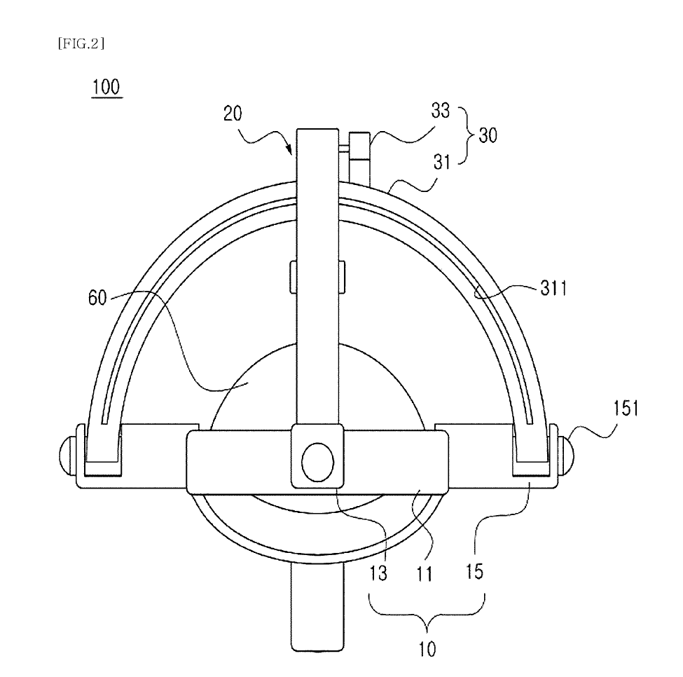

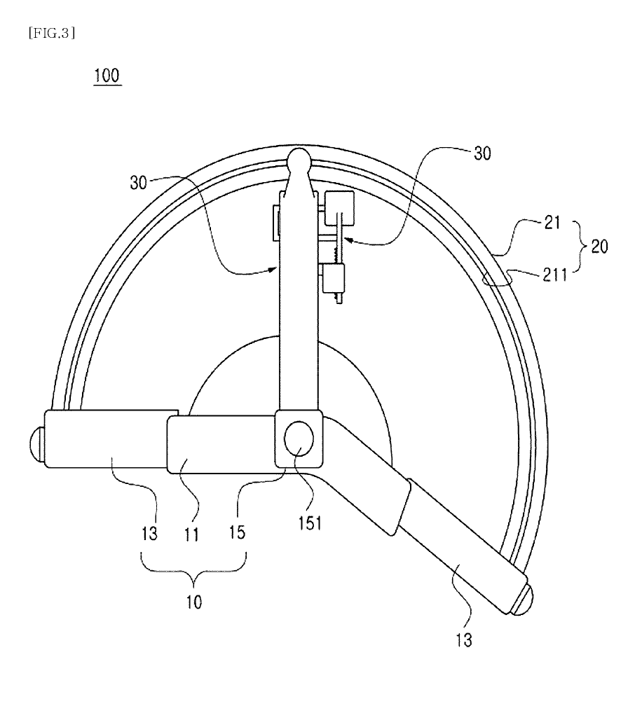

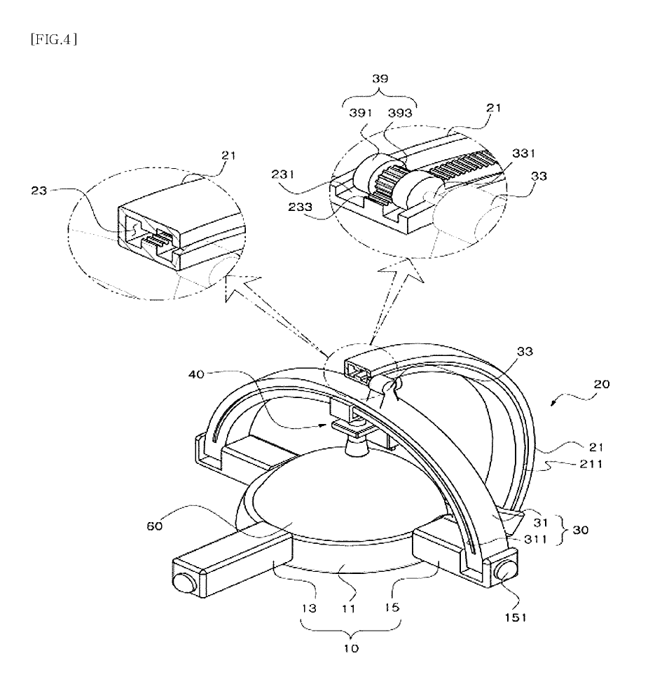

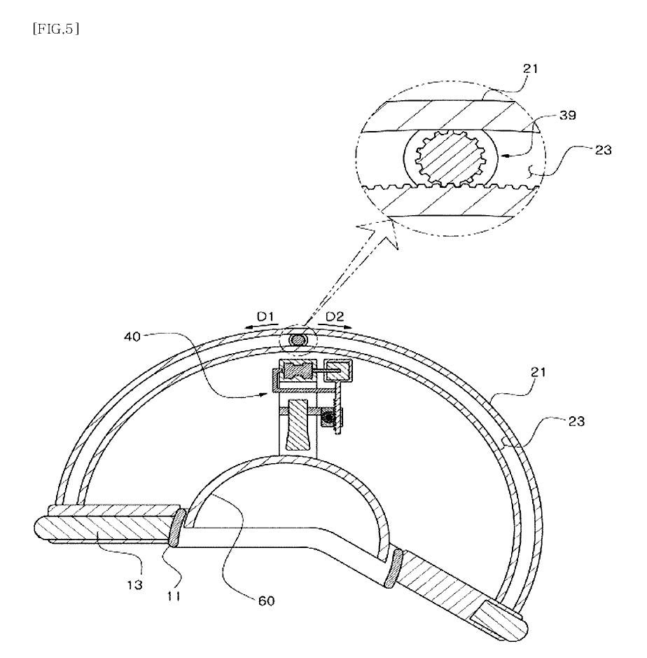

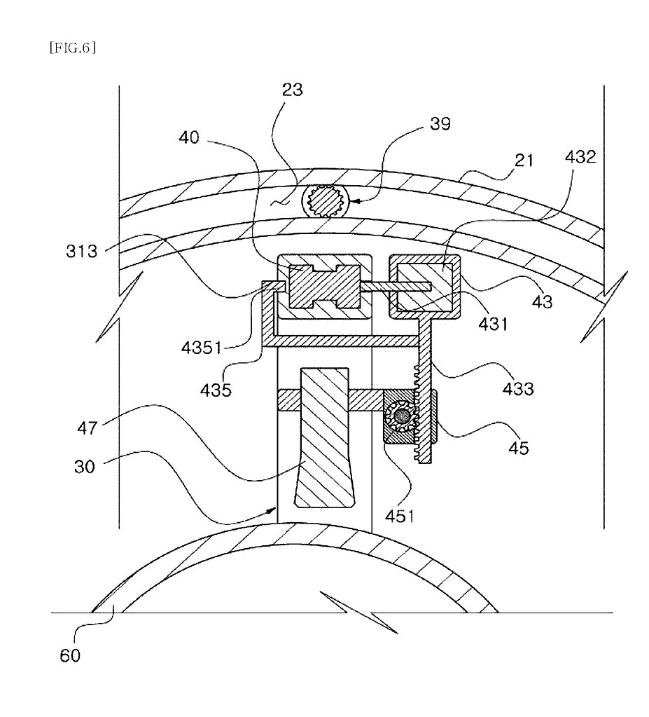

A helmet-type low-intensity focused ultrasound stimulation device according to an exemplary embodiment of the present invention will be described with reference to FIGS. 1 to 6. FIG. 1 is a perspective view illustrating a helmet-type low-intensity focused ultrasound stimulation device according to an exemplary embodiment of the present invention, FIG. 2 is a front view illustrating the helmet-type low-intensity focused ultrasound stimulation device according to the exemplary embodiment of the present invention, and FIG. 3 is a side view illustrating the helmet-type low-intensity focused ultrasound stimulation device according to the exemplary embodiment of the present invention. Further, FIG. 4 is a partially exploded perspective view illustrating a part of the helmet-type low-intensity focused ultrasound stimulation device according to the exemplary embodiment of the present invention, FIG. 5 is a longitudinal cross-sectional view taken along line A-A of FIG. 1, and FIG. 6 is a longitudinal cross-sectional view for describing an ultrasound module.

The helmet-type low-intensity focused ultrasound stimulation device 100 according to the exemplary embodiment of the present invention includes a support part 10, a transmitting unit 60, a first guide part 20, a second guide part 30, and an ultrasound module 40.

Referring to FIGS. 1 to 3, the support part 10 includes a support part body 11, a longitudinally extended arm 13, and a transversely extended arm 15. The support part body 11 is formed in a circular band shape and is mounted on a head of a body. In this case, a rear side of the support part body 11 may be formed in a shape bent in a down direction in order to secure an ultrasound radiating area of an occipital area in the head of the body. The longitudinally extended arm 13 is radially extended in each of a front surface and a rear surface of the support part body 11. Further, the transversely extended arm 15 is radially extended in each of both side surfaces of the support part body 11.

The first guide part 20 includes a first guide part body 21. The first guide part body 21 is formed in an arc shape, and both ends of the first guide part body 21 are fixed to end sides of the longitudinally extended arm 13. A first incised part 211, which is longitudinally incised, is formed in one side surface of the first guide part body 21.

The second guide part 30 includes a second guide part body 31. The second guide part body 31 is formed in an arc shape, and both ends of the second guide part body 31 are fixed to end sides of the transversely extended arm 15. That is, the second guide part 30 is rotated in a longitudinal direction that is a longitudinal direction of the first guide part 20 based on a rotation shaft 151. A guide recess 311 is formed on one side surface of the second guide part body 31 in a longitudinal direction of the second guide part body 31.

An extended part 33 is formed at a center side of an upper surface of the second guide part 30. The extended part 33 is formed in a shape protruding from the upper surface of the second guide part 30 in an up direction, and is guided in the longitudinal direction in a state of being connected to the first guide part 20 to rotate the second guide part 30 in the longitudinal direction.

Referring to FIGS. 4 and 5, an inside space 23 is formed inside the first guide part body 21. A longitudinal section of the inside space 23 is formed in a shape of “H”. Both lateral lower surfaces of the inside space 23 are formed with a guide rail 231 which is stepped to be lower than a center portion, and a rack gear 233 is formed at a center portion of the guide rail 231.

In the meantime, a first motor (not illustrated) is provided at an inner side of the extended part 33, and a first rotation shaft 331 extended from the first motor is introduced into the inside space 23 through the first incised part 211. The first rotation shaft 331 is connected to a first pinion gear 39. The first pinion gear 39 is formed with cylindrical wheels 391 at both sides thereof so as to be movable while rotating along the guide rail 231, and a gear part 393, of which a radius is smaller than that of the wheel 391, and which is formed with gear teeth on an outer peripheral surface thereof, is provided at a center portion of the wheel 391. The gear part 393 moves while rotating in a state of being engaged with the rack gear 233 of the inside space 23.

That is, when the first motor within the extended part 33 is rotated, the first pinion gear 39 is rotated. When the first pinion gear 39 is rotated, the first pinion gear 39 itself moves along the first rack gear 233, so that the extended part 33 moves along the longitudinal direction (D1 or D2) of the first guide part 20.

An inside structure of the second guide part 30 is the same as the inside structure of the first guide part 20. However, the second guide part 30 is different from the first guide part 20 in that the guide recess 311 is further formed on an opposite side of the surface, on which the incised part is formed. The guide recess 311 serves to improve structural stability so that the ultrasound module 40 is movable while being sufficiently supported.

Referring to FIG. 6, the second guide part 30 is also provided with a second pinion gear 49 at an inner side thereof. The second pinion gear 49 is connected with a second motor 432 provided within a fixing part 43 through a rotation shaft 431, and is rotated together with the second motor 432 when the second motor 432 rotates. The second pinion gear 49 moves in the longitudinal direction of the second guide part 30, that is, the transverse direction, by the same manner as that of the first pinion gear 39.

In the meantime, for convenience of the description, hereinafter, the fixing part 43, a distance adjusting unit 45, and a transducer 47 are commonly called an ultrasound module.

As described above, the second guide part 30 is formed with the guide recess 313 on the other surface of the fixing part 43. A third rack gear 433, which is extended in a down direction, is formed at a lower end of the fixing part 43. Further, a fixing part extended art 435, which is extended from an upper portion of the rack gear 433 to the second guide part 30, and the fixing part extended part 435 is extended to pass through a lower portion of the second guide part 30 and is formed with a protruding portion 4351 accommodated in the guide recess 313. The protruding portion 4351 moves in a state of being accommodated in the guide recess 3113, so that it is possible to improve structural stability of the fixing part 43.

The distance adjusting unit 45 includes a third pinion gear 451, and a third motor (not illustrated) connected with the third pinion gear 451 through a rotation shaft at an inner side thereof. When the third motor is rotated, the third pinion gear 451 is rotated, and the distance adjusting unit 45 moves up and down, that is, in an inside direction or an outside direction of the helmet-type low-intensity focused ultrasound stimulation device according to the present exemplary embodiment, along the third rack gear 433 according to the rotation of the third pinion gear 451.

The transducer 47 generates ultrasound by converting electricity into vibration energy. The transducer 47 allows the ultrasound to be emitted in a down direction of the drawing, that is, the inside direction of the helmet-type low-intensity focused ultrasound stimulation device according to the present exemplary embodiment. Further, the transducer 47 is fixed to the distance adjusting unit 45 and moves together with the distance adjusting unit 45 in a direction, in which the distance adjusting unit 45 moves.

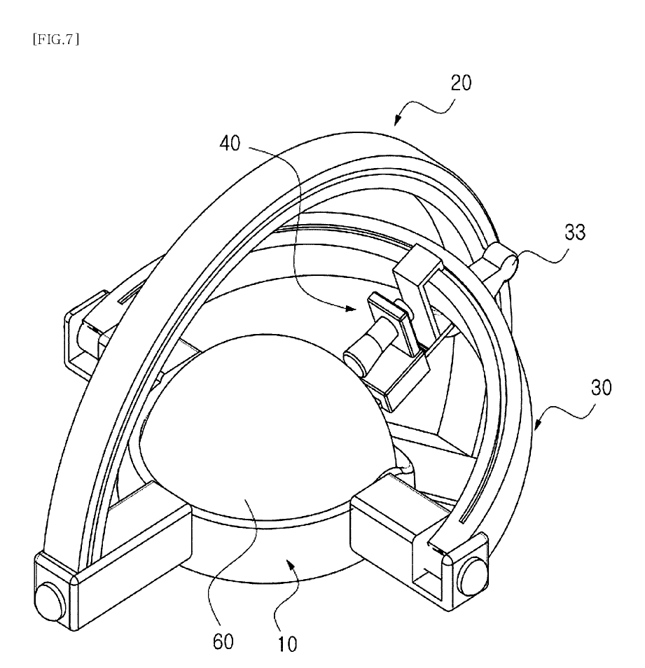

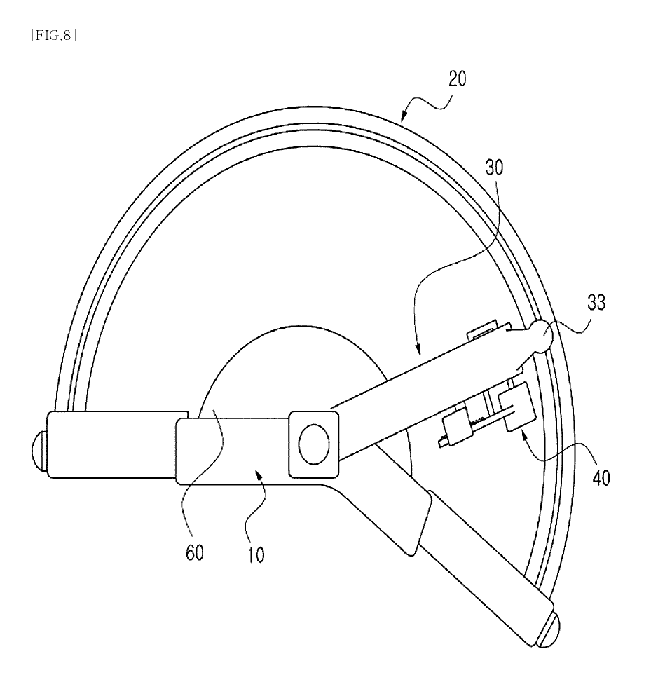

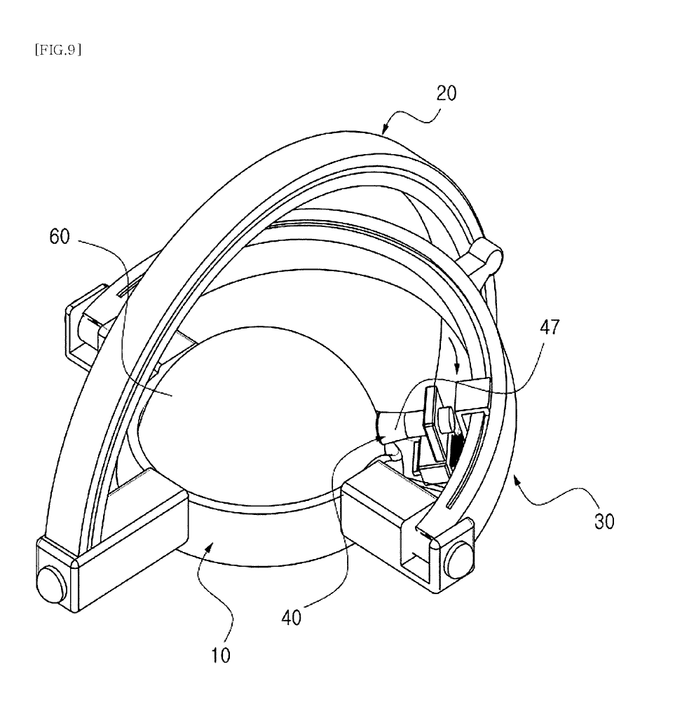

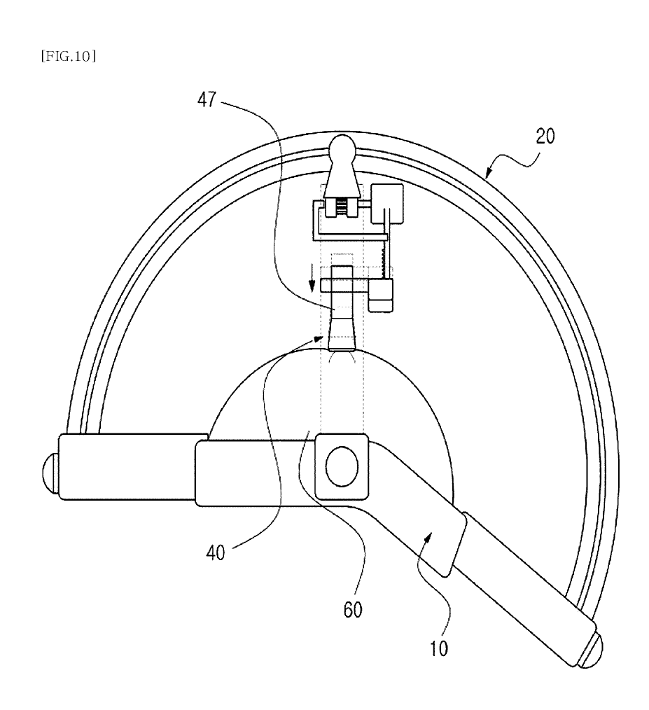

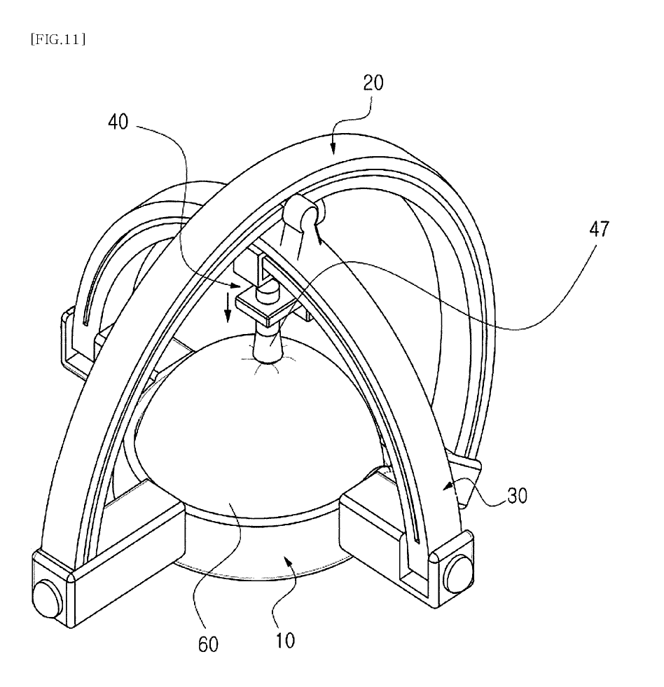

A location control method of the transducer will be described with reference to FIGS. 7 to 11. FIG. 7 is a side view illustrating the helmet-type low-intensity focused ultrasound stimulation device of FIG. 6, FIG. 8 is a perspective view illustrating a longitudinal location control of the helmet-type low-intensity focused ultrasound stimulation device according to the exemplary embodiment of the present invention, and FIG. 9 is a perspective view illustrating a transverse location control of the helmet-type low-intensity focused ultrasound stimulation device according to the exemplary embodiment of the present invention. Further, FIG. 10 is a diagram schematically illustrating a distance control of the ultrasound module according to the exemplary embodiment of the present invention, and FIG. 11 is a perspective view illustrating a distance control of the ultrasound module according to the exemplary embodiment of the present invention.

As illustrate in FIGS. 7 and 8, in order to move the ultrasound module 40 to a specific position, for example, by a specific latitude, a specific longitude, and a specific distance from the center, the extended part 33 is first controlled to move in the longitudinal direction along the first guide part 20. In this case, the ultrasound module 40 also moves in the longitudinal direction in a state of being fixed to the second guide part 30 and is positioned at a specific latitude.

Then, as illustrated in FIG. 9, the ultrasound module 40 is moved in the transverse direction along the guide part 30. In this case, the ultrasound module 40 moves to a position on a specific longitude desired to be controlled while moving along the specific latitude. A height, that is, a distance from the center, of the transducer 47, is adjusted by controlling the ultrasound module 40 by the aforementioned method.

To particularly describe with reference to FIGS. 10 and 11, the transducer 47 does down and is in contact with the transmitting unit 60. Ultrasound has a characteristic in that when ultrasound is in contact with air, the ultrasound is reflected, so that it is necessary to prevent air from being introduced on an emission path of the ultrasound waves. Accordingly, the transducer 47 is in close contact with the transmitting unit 60.

The ultrasound waves generated in the transducer 47 is transmitted to the transmitting unit 60 and the head of the body through a medium material, for example, de-gas water, inside the transmitting unit 60. The transmitting unit 60 may be formed of a synthetic resin material, such as polyethylene.

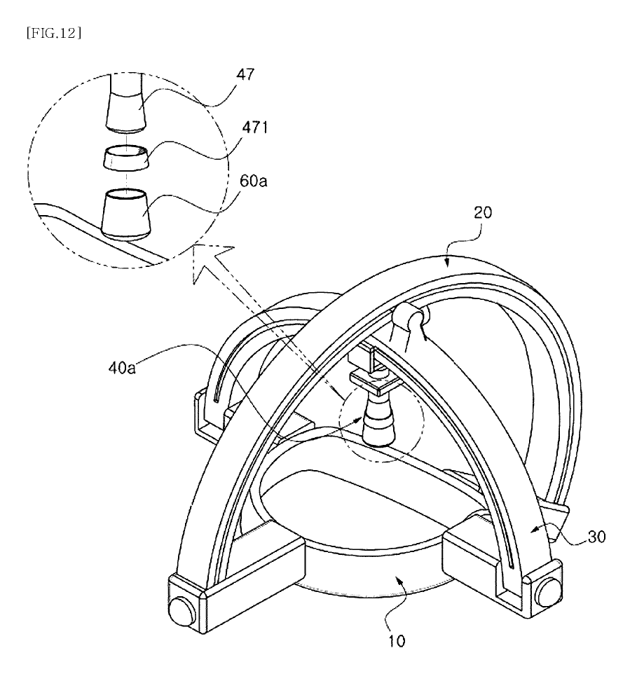

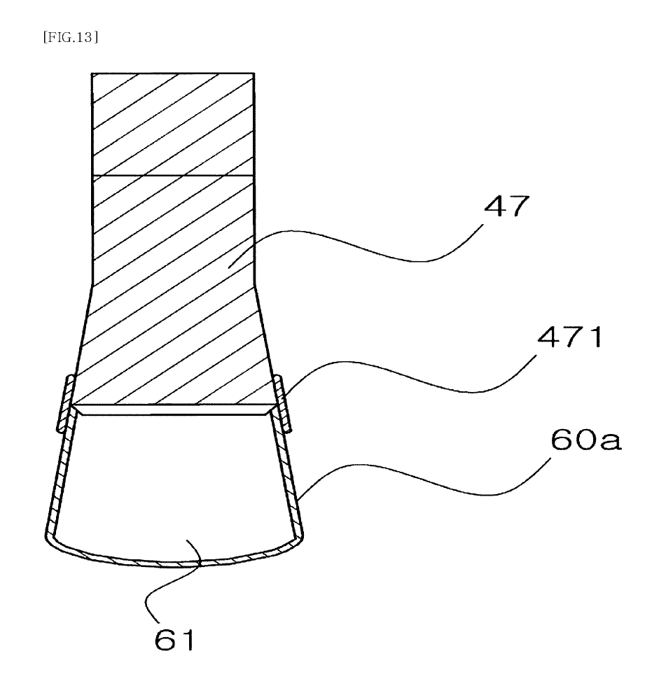

A helmet-type low-intensity focused ultrasound stimulation device according to another exemplary embodiment of the present invention will be described with reference to FIGS. 12 and 13. FIG. 12 is a perspective view illustrating a helmet-type low-intensity focused ultrasound stimulation device including an ultrasound module according to another exemplary embodiment of the present invention, and FIG. 13 is a cross-sectional view illustrating the ultrasound module according to the exemplary embodiment of FIG. 12.

As illustrated in FIGS. 12 and 13, the helmet-type low-intensity focused ultrasound stimulation device according to the present exemplary embodiment is different from the aforementioned exemplary embodiment in a configuration of a transmitting unit 60a. That is, the transmitting unit of the aforementioned exemplary embodiment is directly worn on the head of the body, but the transmitting unit 60a of the present exemplary embodiment is attached to a transducer 47 and moves together with the transducer 47.

Particularly, in a state where the support part 10 is worn on the head of the body, the transmitting unit of the aforementioned exemplary embodiment is not worn.

However, the transmitting unit 60 according to the present exemplary embodiment is attached to a lower end of the transducer 47. The transmitting unit 60a may be fixed to the lower end of the transducer 47 by using a coupler 471.

The transmitting unit 60a moves together with the transducer 47 according to the movement of the transducer 47, and then goes down together with the transducer 47 when the transducer 47 goes down and is in contact with the head of the body. In the meantime, the transmitting unit 60a stores a medium material, such as de-gas water, inside thereof as described above.

Further, ultrasound waves generated by the transducer 47 move to the head of the body through the transmitting unit 60a and the medium material within the transmitting unit 60a by the same method.

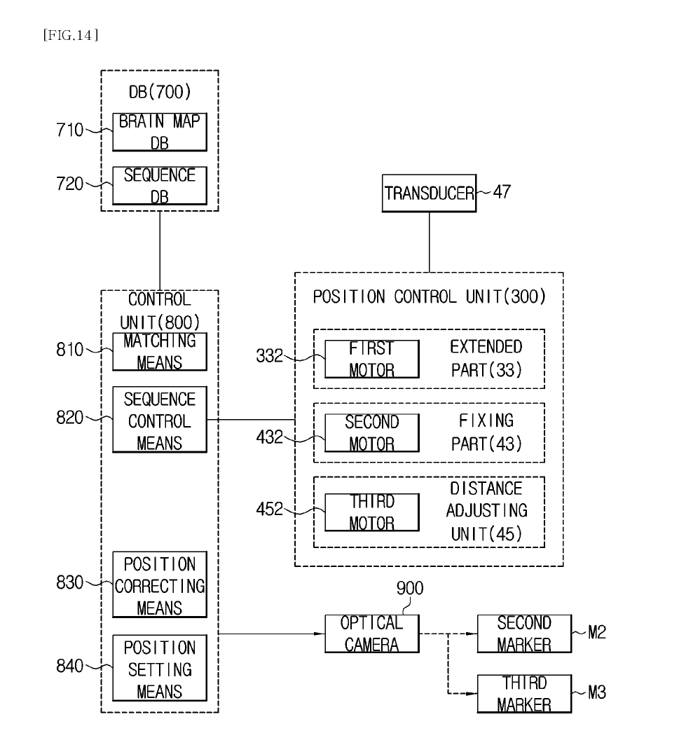

A helmet-type low-intensity focused ultrasound stimulation system according to an exemplary embodiment will be described with reference to FIG. 14. FIG. 14 is a block diagram illustrating a helmet-type low-intensity focused ultrasound stimulation system according to an exemplary embodiment of the present invention.

A database 700 includes a brain map database 710 and a sequence database 720.

A 3D relative coordinate value for each part of a head of a standard human body including a brain is stored in the brain map database 710. For example, a relative coordinate value for each part based on a specific position in a human head shape set as a standard may be stored in the brain map database 710.

The sequence database 720 includes an ultrasound stimulation method, and a relative coordinate value of a specific part of a brain to which the corresponding ultrasound stimulation method is applied. In this case, the ultrasound stimulation method may include an intensity of ultrasound, an ultrasound stimulation time, the number of times of the ultrasound stimulation, a period of the ultrasound stimulation, and the like. Hereinafter, for convenience of the description, a set of the ultrasound stimulation method and a relative coordinate value of an applied part is referred to as sequence data. In the meantime, a plurality of combinations of the set, as well as the set of the ultrasound stimulation method and a relative coordinate value of an applied part, may also correspond to the sequence data. Further, the sequence data may be stored in a form corresponding to a specific treatment, such as relief and a treatment of a specific disease and relief and a treatment of a specific pain.

For example, the sequence data stored in the sequence database may store the performance of a scheme of radiating ultrasound waves with a first intensity to a first position of the brain for about three seconds and having a resting phase for one second by three times, and the performance of a scheme of radiating ultrasound waves with a second intensity to a second position of the brain for about two seconds and having a resting phase for two seconds by two times for treating a hand numb phenomenon as a serial of treatment sequences.

A control unit 800 includes a matching means 810, a sequence control means 820, a position correcting means 830, and a position setting means 840.

The matching means 810 receives image data of a specific patient from medical imaging equipment, such as Computerized Tomography (CT), Magnetic Resonance Imaging (MRI), and Functional Magnetic Resonance Imaging (fMRI), and matches the received image data with the relative coordinate value of the head of the standard human body stored in the brain map database 710. In this case, the matching means 800 receives the image of the patient, who is in a state of being attached with a first marker detectible by the medical imaging equipment, such as CT, MRI, and fMRI.

A brain structure of human is different in a size, a shape, and the like for everyone, so that when ultrasound is radiated by using the relative coordinates of the standard human body, reliability is degraded. Accordingly, a relative coordinate value optimized to an individual is calculated by matching an image of a head of a specific patient photographed by using the medical imaging equipment, such as CT, MRI, and fMRI, with the relative coordinate value of the head of the standard human body.

The sequence control means 820 selects any one of the sequence data, controls a location of the transducer according to the relative coordinate and the stimulation method corresponding to the corresponding sequence data stored in the sequence database, and control an operation of the transducer at a corresponding position. In this case, the selection of any one of the sequence data may be performed by various methods. For example, when an identification number of a specific patient is input, specific sequence data may be automatically selected by inquiring a medical record of the corresponding patient, or a specific sequence may also be directly selected according to a manipulation of a manager.

The sequence control means 820 controls a position of the transducer 47 through the control of a position control unit 300 of a first motor 332, a second motor 432, and a third motor 452 in the helmet-type low-intensity focused ultrasound stimulation device.

The position correcting means 830 obtains positon change information of the helmet-type low-intensity focused ultrasound stimulation device for a head of a patient based on a change of a position of a third marker on an image photographed by an optical camera 900. In this case, the change of the position of the helmet-type low-intensity focused ultrasound stimulation device for the head of the patient means that the helmet, which the patient wears, is mis-located by a movement of the patient or an application or external force. When the helmet is mis-located, an initial position of the transducer 47 is changed, thereby accuracy is degraded. In the meantime, the third marker and the method of obtaining the position change information will be described in detail with reference to the relevant drawing.

The position setting means 840 sets initial coordinates of the transducer 47. A coordinate system forming the relative coordinates of the brain map database 710 needs to be linked with a coordinate system required for controlling the helmet-type low-intensity focused ultrasound stimulation device based on a specific position. That is, when a stimulus is required to a specific part of a brain of a patient, an initial position of the transducer needs to be linked with a specific position in the brain coordinate system by setting the initial position of the transducer in a state where the patient wears the helmet.

The optical camera 900 photographs a second marker and a third marker attached to a head of a patient in a state of being fixed to the helmet-type low-intensity focused ultrasound stimulation device.

In the meantime, the positions, at which the database 700 and the control unit 800 are formed, are not limited. That is, the helmet-type low-intensity focused ultrasound stimulation system is a name for being discriminated from the helmet-type low-intensity focused ultrasound stimulation device corresponding to the mechanical configuration, and is not for discriminating a physical configuration. The database 700 and the control unit 800 may be integrally formed with the helmet-type low-intensity focused ultrasound stimulation device, and may also be implemented in a separate device.

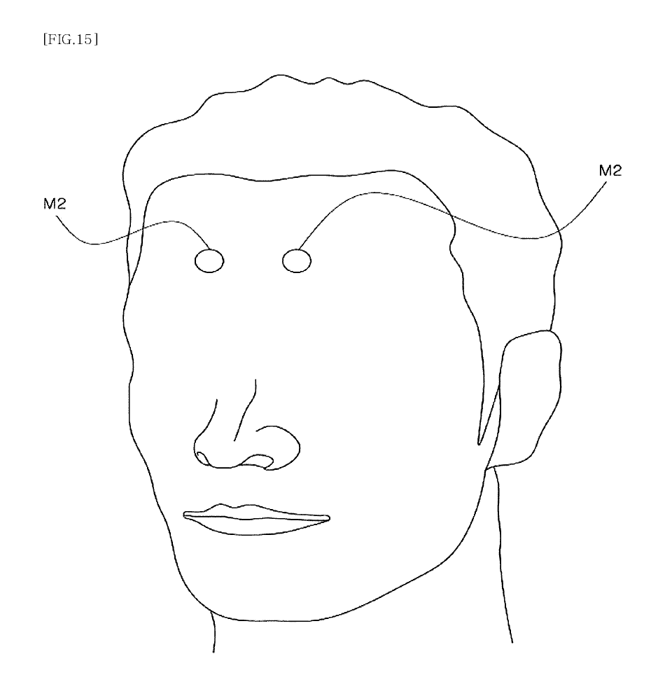

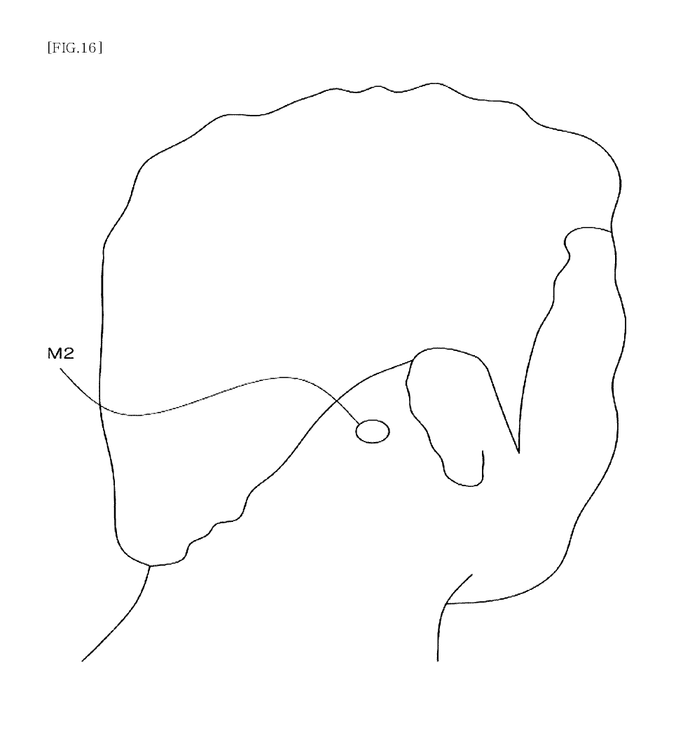

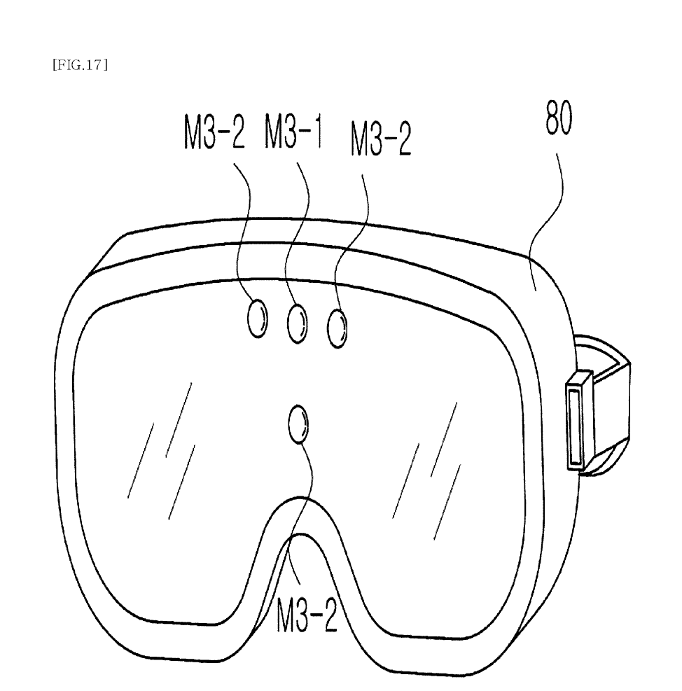

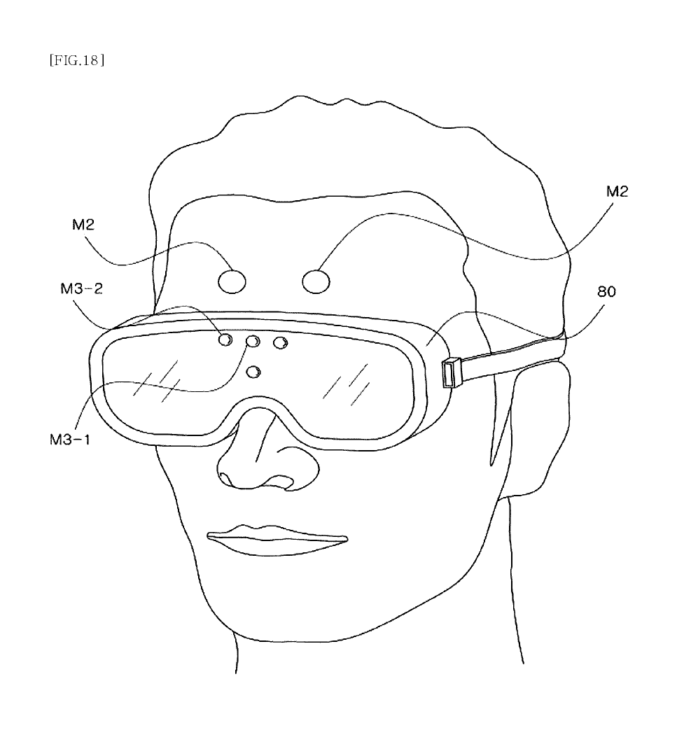

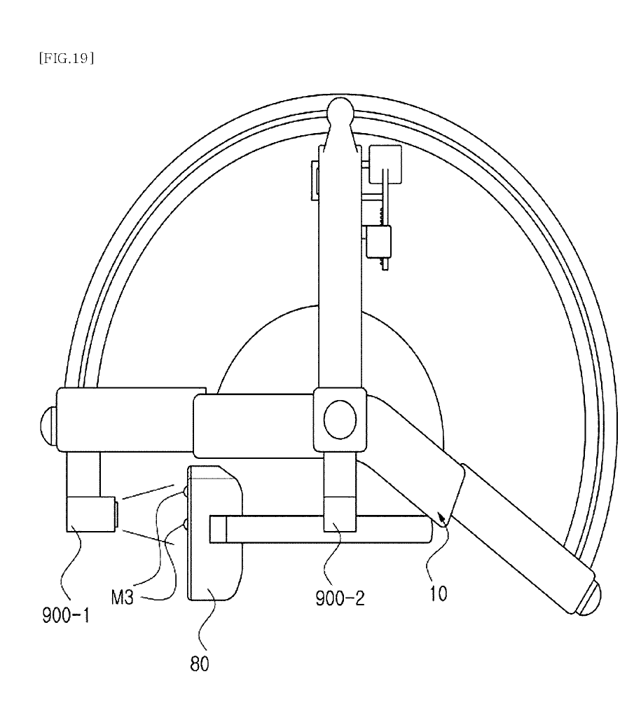

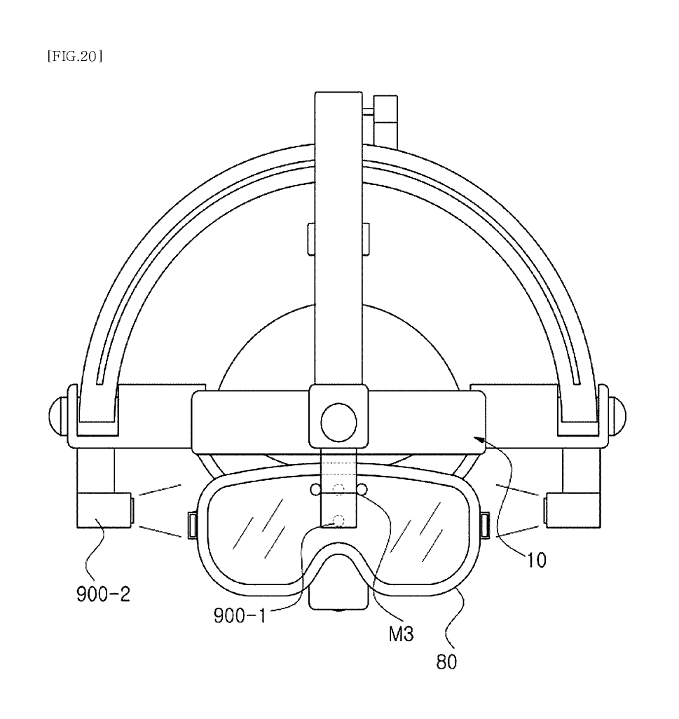

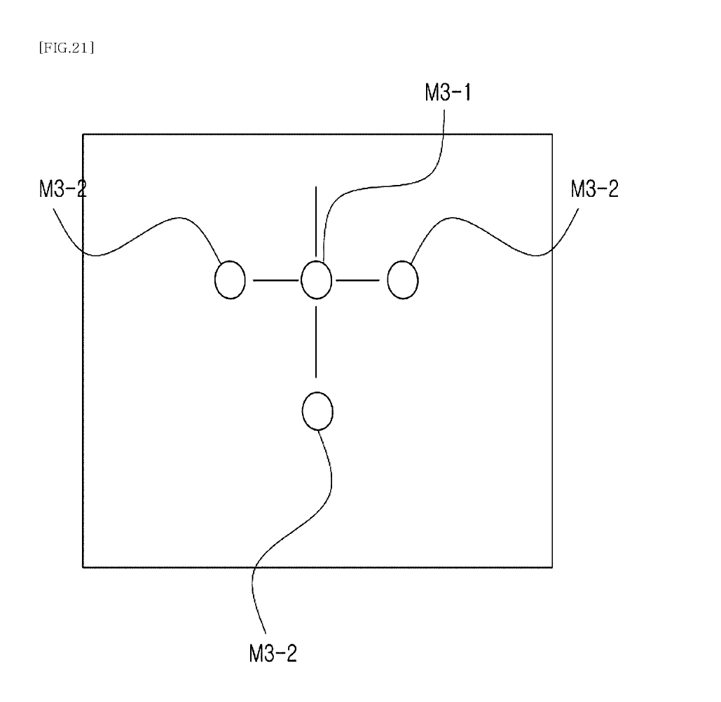

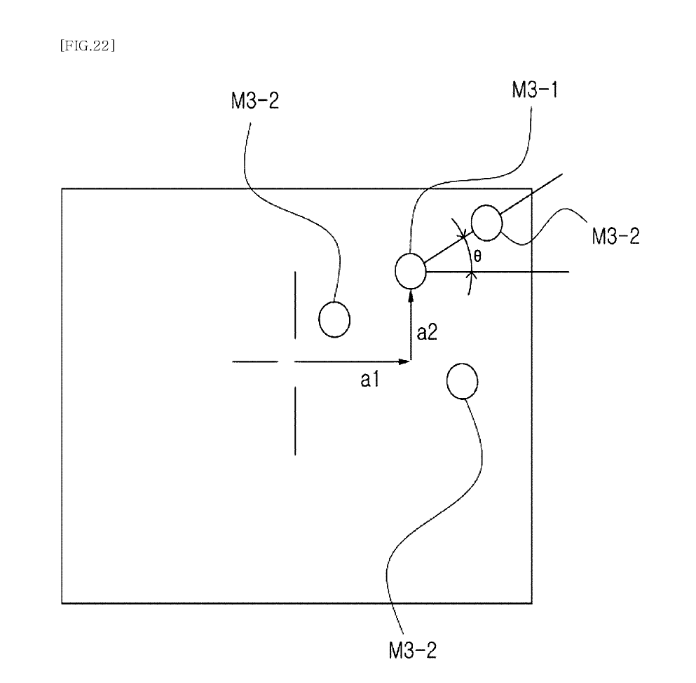

The markers according to the present invention and the method of controlling a position of the transducer by using the markers will be described in detail with reference to FIGS. 15 to 22. FIGS. 15 and 16 are diagrams schematically illustrating an attachment position of the first marker according to the exemplary embodiment, and FIG. 17 is a diagram schematically illustrating goggles, to which the third marker is attached. Further, FIG. 18 is a diagram schematically illustrating a state where a patient wears goggles, FIGS. 19 and 20 are diagrams schematically illustrating the states, in which the third markers in the front surface and the side surface are photographed, and FIGS. 21 and 22 are diagrams schematically illustrating an example of continuous frames of the photographed third marker.

The second marker M2 may be formed of an photoreflective material reflecting light used by a corresponding optical camera. For example, the second marker M2 may be formed of a material reflecting infrared rays so as to reflect infrared rays radiated by an infrared camera and enable the infrared camera to detect the reflected infrared rays. A globular marker, which is capable of uniformly reflecting light in any direction, is used as a marker generally used in a motion capture and the like, but in a case of the present exemplary embodiment, a position variation between the optical camera and the marker is not large, so that any kind of marker having a protruding shape with a uniformly curved surface, such as a semi-spherical shape, and a semi-cylindrical shape, may be used.

A plurality of second markers M2 may be provided on a forehead of a patient as illustrated in FIG. 15, and may be attached to the back sides of both ears, that is, occipital areas adjacent to the ears as illustrated in FIG. 16.

In the meantime, the second marker M2 needs to be attached to the position of the first marker, which is attached when a head image of the patient is photographed by using medical equipment, such as CT, MRI, and fMRI. In a case of the present exemplary embodiment, the first marker needs to be attached to a position including an attachment position of the second marker M2 illustrated in FIGS. 15 and 16. The first marker may be attached to each of the top of the head, a forehead, and an occipital area adjacent to an ear of the patient, so that the second marker may also be attached to the positions of the top of the head, a forehead, and an occipital area adjacent to an ear of the patient according to the position of the first marker, but it is possible to easily perform the photographing by the optical camera according to the present exemplary embodiment, so that the top of the head may be excluded.

As described above, the matched coordinates of the brain of the patient and the coordinate system for controlling the transducer need to be linked based on a specific point. In this case, the first marker and the second marker serve as specific points for linking the two coordinate systems.

In the meantime, the third marker is used for the purpose of correcting a position. The aforementioned second marker may be used as the third marker. Further, a separate marker attached to the patient may also be used as the third marker, and as illustrated in FIG. 17, the markers M3-1 and M3-2 attached onto the goggles 80, which the patient is wearable, may also be used.

Further, as illustrated in FIG. 18, the newly attached third markers M3-1 and M3-2 may also be used for the purpose of correcting a position together with the second marker M2.

In the meantime, the optical cameras 900-1 and 900-2 may be attached onto a front surface and both side surfaces of the support part 10 as illustrated in FIGS. 19 and 20. The front optical camera 900-1 may photograph the third marker M3 attached onto the goggles 80 or the second marker attached onto the forehead of the patient. Further, the optical cameras 900-2 attached to both side surfaces may photograph the second markers attached to the occipital areas adjacent to the ears of the patient.

At least two markers need to be used as the third markers. Any one M3-1 of the third markers is used as a reference marker for calculating a movement distance of a specific point, and the remaining third markers M3-2 are used for calculating a rotation angle based on the reference marker.

As described above, the position correcting means continuously photographs the third marker and detects the movement of the third marker, thereby correcting an error by a mis-location of the helmet-type low-intensity focused ultrasound stimulation device worn on the patient. Particularly, the position correcting means determines whether the positions of the third markers are changed between the continuously photographed frames as illustrated in FIGS. 21 and 22, and when the positions of the third markers are changed, the position correcting means calculates a movement distance (R(a1, a2) of the reference marker M3-1 among the third markers and a rotation angle q of the remaining third markers M3-2 based on the reference marker, thereby calculating an error generated due to the movement of the helmet.

In general, when the movements of the markers based on the camera are large, swapping of the markers may be incurred. That is, when the positions of the markers are reverse between the continuous frames, it may be difficult to identify the two markers. However, in the case of the present exemplary embodiment, the position change of the markers in a state where the patient wears the helmet-type low-intensity focused ultrasound stimulation device is not severe enough to reverse the positions of the markers, so that it is possible to simply use only two markers, and a separate marker identification is not required.

The position correcting means may transmit the calculated movement distance of the reference marker and the rotation angle based on the reference marker to the position setting means to reset the initial coordinates of the transducer, or may transmit the calculated movement distance of the reference marker and the rotation angle based on the reference marker to the sequence control means to reflect the calculated error to the control of the position of the transducer.

In the above, the exemplary embodiments of the present invention have been described, but the technical spirit of the present invention is not limited to the aforementioned exemplary embodiments, and various modifications may be made within the scope of the technical spirit of the present invention embodied in the claims.

1. A helmet-type low-intensity focused ultrasound stimulation device, comprising:
a first guide part which is formed in an arc shape and is provided in a longitudinal direction;
a second guide part which is formed in an arc shape and is connected in a transverse direction so as to be orthogonal to the first guide part;
an ultrasound module which is connected to the second guide part and includes a transducer for generating ultrasound waves moving towards an inner direction; and
a support part which is worn on a head of a user, and to which the first guide part is fixed and both end portions of the second guide part are rotationally fixed,
wherein the second guide part rotates in the longitudinal direction by allowing one point thereof to be guided along the first guide part, and
the transducer is movable along the second guide part in a transverse direction.

2. The helmet-type low-intensity focused ultrasound stimulation device of claim 1, wherein the ultrasound module includes a distance adjusting unit which moves the transducer in the moving direction of the ultrasound waves or in a reverse direction.

3. The helmet-type low-intensity focused ultrasound stimulation device of claim 2, wherein the first guide part is formed with a first rack gear in the longitudinal direction, and
the second guide part is provided with a first pinion corresponding to the first rack gear.

4. The helmet-type low-intensity focused ultrasound stimulation device of claim 3, wherein the second guide part includes an extended part which protrudes in an upper direction and includes a first motor driving the first pinion at an inner side thereof.

5. The helmet-type low-intensity focused ultrasound stimulation device of claim 3, wherein the second guide part is formed with a second rack gear in the longitudinal direction, and
the ultrasound module includes a second pinion corresponding to the second rack gear.

6. The helmet-type low-intensity focused ultrasound stimulation device of claim 5, wherein the ultrasound module includes a fixing part which includes a second motor driving the second pinion at an inner side thereof.

7. The helmet-type low-intensity focused ultrasound stimulation device of claim 6, wherein the distance adjusting unit includes:
a third rack gear formed from the fixing part in an inner direction;
a third pinion which moves in the inner direction or an outer direction in response to the third rack gear and adjusts a radial distance of the transducer; and
a third motor which drives the third pinion.

8. The helmet-type low-intensity focused ultrasound stimulation device of claim 1, further comprising:
a transmitting unit which stores an ultrasound medium material at an inner side thereof, and is interposed between the head of the user and the transducer to mediate ultrasound transmission.

9. The helmet-type low-intensity focused ultrasound stimulation device of claim 8, wherein the transmitting unit is fixed to a lower end of the transducer.

10. The helmet-type low-intensity focused ultrasound stimulation device of claim 8, wherein the transmitting unit is formed of a synthetic resin material.

11. The helmet-type low-intensity focused ultrasound stimulation device of claim 8, wherein the medium material is de-gas water.

12. A helmet-type low-intensity focused ultrasound system, comprising:
a brain map database which stores a 3D relative coordinate value for each part of a head of a standard human body including a brain;
a sequence database which stores an ultrasound stimulation method including at least one of an intensity of ultrasound stimulation, an ultrasound stimulation time, the number of times of the ultrasound stimulation, a period of the ultrasound stimulation, and sequence data related to a stimulation control including a relative coordinate value of a specific part of a brain to which the ultrasound stimulation method is to be applied;
a helmet-type low-intensity focused ultrasound device which includes a transducer for generating ultrasound waves, and a support part mounted on a head of a patient and supporting the transducer to have a position movement on the head of the patient; and
a sequence control means which controls a position of the transducer and controls an operation of the transducer at the corresponding position according to relative coordinates and a stimulation method corresponding to corresponding sequence data stored in the sequence database by selecting any one from among the sequence data.

13. The helmet-type low-intensity focused ultrasound stimulation system of claim 12, wherein the sequence database further includes sequence data formed of a combination of sets including the ultrasound stimulation method and the relative coordinate value.

14. The helmet-type low-intensity focused ultrasound stimulation system of claim 13, wherein the sequence data is stored in the sequence database in correspondence with a specific treatment including any one of relief and a treatment of a specific disease and relief and a treatment of a specific pain.

15. The helmet-type low-intensity focused ultrasound stimulation system of claim 14, wherein the sequence control means receives data for a specific treatment from the outside, inquires sequence data corresponding to the specific treatment in the sequence database, and controls a position and an operation of the transducer according to the inquired sequence data.

16. The helmet-type low-intensity focused ultrasound stimulation system of claim 12, further comprising:
a matching means which receives image data of the head of the patient from an external device performing any one photographing method selected from Computerized Tomography (CT), Magnetic Resonance Imaging (MRI), and Functional Magnetic Resonance Imaging (fMRI), and matches the received image data with the 3D relative coordinate value of the head of the standard human body stored in the brain map database.

17. The helmet-type low-intensity focused ultrasound stimulation system of claim 16, wherein the sequence control means controls a position of the transducer by using a relative coordinate value, which is converted so as to correspond to the head of the patient by the matching means.

18. A helmet-type low-intensity focused ultrasound stimulation system, comprising:
a helmet-type low-intensity focused ultrasound stimulation device which includes a transducer for generating ultrasound waves, and a support part mounted on a head of a patient and supporting the transducer to have a position movement on the head of the patient;
a position setting means which sets initial coordinates of the transducer;
a sequence control means which performs a position control based on the initial coordinates of the transducer, and performs a control according to an ultrasound stimulation method including at least one of an intensity of a stimulation, a stimulation time, the number of times of the stimulation, a period of the stimulation of ultrasound waves generated by the transducer;
one or more optical cameras which is fixed to the helmet-type low-intensity focused ultrasound stimulation device, and photographs third markers attached onto the head of the patient; and
a position correcting means which obtains position change information of the helmet-type low-intensity focused ultrasound stimulation device for the head of the patient based on a position change of the third marker on an image photographed by the optical camera.

19. The helmet-type low-intensity focused ultrasound stimulation system of claim 18, further comprising:
a brain map database which stores a 3D relative coordinate data for each part of a head of a standard human body including a brain; and
a matching means which receives image data of the head of the patient from an external device performing any one photographing method selected from Computerized Tomography (CT), Magnetic Resonance Imaging (MRI), and Functional Magnetic Resonance Imaging (fMRI), and matches the received image data with the 3D relative coordinate value of the head of the standard human body stored in the brain map database,
wherein the position setting means sets initial coordinates of the transducer by linking the matched coordinate system of the image data of the head of the patient with a coordinate system for a control area of the transducer.

20. The helmet-type low-intensity focused ultrasound stimulation system of claim 19, wherein the matching means receives the image data of the head of the patient, to which a plurality of first markers detectible by the selected photographing method is attached, from the external device,
the optical camera photographs a second marker provided at a position of the first marker, and
the position setting means links the matched coordinate system of the image data of the head of the patient and the coordinate system for the control area of the transducer based on the positions of the first marker and the second marker.

21. The helmet-type low-intensity focused ultrasound stimulation system of claim 20, wherein the position setting means calculates a movement distance of any one marker, which is selected as a reference marker among the third markers, according to a position change between specific frames of an image photographed by the optical camera, and calculates a rotation angle of one or more markers, which are not selected as the reference marker among the third markers, based on the reference marker.

22. The helmet-type low-intensity focused ultrasound stimulation system of claim 21, wherein the position correcting means transmits the calculated movement distance of the reference marker and the calculated rotation angle based on the reference marker to the position setting means, and
the position setting means resets the initial coordinates of the transducer by reflecting the received movement distance of the reference marker and the received rotation angle based on the reference marker.

23. The helmet-type low-intensity focused ultrasound stimulation system of claim 21, wherein the position correcting means transmits the calculated movement distance of the reference marker and the calculated rotation angle based on the reference marker to the sequence control means, and
the sequence control means reflects the received movement distance of the reference marker and the received rotation angle based on the reference marker during a position control of the transducer.

24. The helmet-type low-intensity focused ultrasound stimulation system of claim 20, wherein the second marker and the third marker are photoreflective.

25. The helmet-type low-intensity focused ultrasound stimulation system of claim 20, wherein the first marker is attached to each of the top of the head, a forehead, an occipital area adjacent to an ear of the patient.

26. The helmet-type low-intensity focused ultrasound stimulation system of claim 25, wherein the second marker is attached to each of the forehead and the occipital area adjacent to the ear at least.

27. The helmet-type low-intensity focused ultrasound stimulation system of claim 26, wherein the second marker is attached onto the first marker after an image is photographed by the external device.

28. The helmet-type low-intensity focused ultrasound stimulation system of claim 26, wherein the third markers include two or more markers selected from the second marker.
